SEQUENCE LISTING (1 sequence)

SEQ ID NO: 1 - DNA, 23 bp, Artificial sequence
  Animal (for example mouse, rat, dog, monkey, or ape)
cctgtatcacatgcggctctcgg

(19) United States
(12) Patent Application Publication
(10) Pub. No.: US 2020/0375157 A1
(43) Pub. Date: Dec. 3, 2020

(54) CONSTRUCTION METHOD FOR ANIMAL MODEL FOR RETINITIS PIGMENTOSA DISEASES AND APPLICATION

(71) Applicant: SICHUAN PROVINCIAL PEOPLE'S HOSPITAL, Chengdu, Sichuan (CN)

(72) Inventors: Xianjun ZHU, Chengdu (CN); Zhenglin YANG, Chengdu (CN); Lin ZHANG, Chengdu (CN); Yeming YANG, Chengdu (CN)

(21) Appl. No.: 16/613,755

(22) PCT Filed: Nov. 30, 2018
(86) PCT No.: PCT/CN2018/118431

§ 371 Date: Nov. 14, 2019

(30) Foreign Application Priority Data
Dec. 4, 2017 (CN) 201711265100.7

(51) Int. Cl. A01K 67/027; C12N 9/12; C12N 15/11; C12N 9/22

(52) U.S. Cl. CPC A01K 67/0276; C12N 9/1205; C12Y 207/01001; C12N 15/11; C12N 2800/80; A01K 2217/075; A01K 2227/105; A01K 2267/0306; C12N 2310/20; C12N 9/22

(57) ABSTRACT

Provided is a construction method for an animal model for retinitis pigmentosa diseases and the application thereof, which relate to the technical field of medical engineering. According to the construction method for the animal model for retinitis pigmentosa diseases, by knocking out Hkdc1 gene sequences on genomes of target animals, the animal model for retinitis pigmentosa diseases is obtained. The animal model presents typical characteristics of retinitis pigmentosa, such as retina function damage and gradual apoptosis of photoreceptor cells. The animal model can be applied to research of retinitis pigmentosa diseases and for to screening medicine for the treatment of retinitis pigmentosa diseases.

The application is a 371 of international application number PCT/CN2018/118431, filed on Nov. 30, 2018, which claims the priority to the Chinese patent application with the filing number 201711265100.7, filed on Dec. 4, 2017 with the Chinese Patent Office and entitled “Construction Method for Animal Model for Retinitis Pigmentosa Diseases and Application.” Each of the aforementioned applications are hereby incorporated by reference in their entirety.

### technical field

The present disclosure relates to the technical field of medical engineering, in particular to a construction method for an animal model with retinitis pigmentosa diseases and application.

### BACKGROUND ART

Retinitis Pigmentosa (RP) is a group of ocular diseases of pathological changes and death of degenerative retinal photoreceptor cells with relatively strong heterogeneity, and at the beginning of morbidity, retinal rod cells are generally first affected, causing declination of scotopic vision, and peripheral visual field is damaged, and a tubular visual field is gradually developed till blindness; retinal pigmentation can be seen in fundus inspection. The morbidity rate of RP in China is about 1/4000, and based on huge population base of China, there may be millions of RP patients, bringing about heavy loads to families and society. More unfortunately, in-depth and systematic research of cause and pathological mechanism of the diseases currently is lacked, and pathogenic mechanisms of considerable RP genes remain unknown, bringing about an obstacle to targeted development of treating and intervening measures.

A root cause of weak RP control measures lies in lack of in-depth and systematic research of cause and pathological mechanism of the diseases. Such dilemma is mainly caused by following aspects: 1) clinical phenotype heterogeneity is strong. The clinical phenotypes of this disease are extremely different. A severe patient starts to be attacked by the disease in adolescence, and goes blind very soon, while a moderate patient still can have part of the visual function in middle age. This characteristic of RP presents a huge challenge to conventional diagnosis methods. 2) There are many virulence genes and diversified genetic modes. There are now over 70 known RP genes, which can explain about 60% of RP cases, but there are still nearly 40% of the cases cannot be explained. Meanwhile, genetic modes of this disease are diversified, including a plurality of genetic modes such as autosomal dominance, recessive inheritance, X-linked inheritance, and mitochondrial inheritance. Therefore, looking for new virulence genes and knowing their pathogenic mechanism are foremost problems to be settled for research of this disease. 3) The pathogenic molecular mechanism is complex. Already discovered cellular biochemical processes in which RP pathogenic genes participate are quite complex, for example, photosignal transduction, cytoskeleton, RNA splicing, ubiquitylation degradation and a variety of biochemical processes, which causes it hard to research the mechanism of pathogenic molecules thereof, and also brings about many difficulties to treatment and intervention.

At present, however, animal models with retinitis pigmentosa diseases are still lacked, inconveniencing further in-depth and systematic research of retinitis pigmentosa diseases.

In view of this, the present disclosure is specifically proposed.

### SUMMARY

An object of the present disclosure includes, but is not limited to, providing a construction method for an animal model with retinitis pigmentosa diseases, and the animal model with retinitis pigmentosa diseases can be obtained according to this construction method, and can be used for researchers' in-depth and systematic research of retinitis pigmentosa diseases, and for screening a medicine for the retinitis pigmentosa diseases.

Another object of the present disclosure includes, but is not limited to, providing an application of the animal model with retinitis pigmentosa diseases obtained through the above construction method in research of retinitis pigmentosa diseases.

Another object of the present disclosure includes, but is not limited to, providing a nucleic acid molecule for constructing an animal model with retinitis pigmentosa diseases.

The present disclosure is realized as follows:

A construction method for an animal model with retinitis pigmentosa diseases, which includes: knocking out a sequence of interest, which is Hkdc1 gene, on a genome of a target animal, to obtain a founder animal with the Hkdc1 gene being knocked out.

Application of the animal model with retinitis pigmentosa diseases obtained through the above construction method for an animal model with retinitis pigmentosa diseases in screening a medicine for treating retinitis pigmentosa diseases.

Application of the animal model with retinitis pigmentosa diseases obtained through the above construction method for an animal model with retinitis pigmentosa diseases in research of retinitis pigmentosa diseases.

A nucleic acid molecule for constructing an animal model with retinitis pigmentosa diseases, wherein the nucleic acid molecule is gRNA, and a base sequence of a target site to which this gRNA is directed is represented by SEQ ID NO:1

The Present Disclosure has Following Beneficial Effects:

According to the construction method for an animal model with retinitis pigmentosa diseases provided in the present disclosure, by knocking out the Hkdc1 gene on the genome of the target animal, the animal model presenting typical characteristics of retinitis pigmentosa, such as retina function damage and gradual apoptosis of photoreceptor cells, can be obtained, and this animal model can be used for research of retinitis pigmentosa diseases and for screening a medicine for treating retinitis pigmentosa diseases, which has a broad application prospect.

### BRIEF DESCRIPTION OF DRAWINGS

In order to more clearly illustrate technical solutions of examples of the present disclosure, accompanying drawings which need to be used in the examples will be introduced below briefly. It should be understood that only some examples of the present disclosure are shown in the accompanying drawings below, and therefore should not be considered as limitation on the scope. A person ordinarily skilled in the art still can obtain other relevant accompanying drawings according to these accompanying drawings, without paying inventive efforts.

FIG. 1 is a design schematic diagram of a CRISPR targeting sequence provided in an example of the present disclosure;

FIG. 2 shows diagram of genotype identification for Hkdc1 knockout mice provided in an example of the present disclosure; (Mut: NM_145419.1 , c.93_99delCTCGGATG, in the drawings, WT represents wild type, Hom represents gene knockout homozygous genotype, and Het represents gene knockout heterozygous genotype);

FIG. 3 shows WB results provided in an example of the present disclosure;

FIG. 4 shows IHC staining results provided in an example of the present disclosure (in the drawing: WT is a wild-type mouse, and Mut/Mut is a homozygous mouse with the Hkdc1 gene being knocked out);

FIG. 5 shows a result of faultily positioning retinal rhodopsin proteins of KO mice, provided in an example of the present disclosure, in inner segment and soma of a photoreceptor cell (the mice are 9 months old; arrows indicate the faultily positioned rhodopsin proteins); and

FIG. 6 shows retina HE staining results of 11-month and 17-month mice (in the drawings, A and B respectively represent hematoxylin-eosin staining results (H&E staining) of 11-month wild-type and mutant-type retinal sections; C represents a statistical result of the number of rows of 11-month photoreceptor cells; D and E respectively represent hematoxylin-eosin staining results (H&E staining) of 17-month wild-type and mutant-type retinal sections; and F represents a statistical result of the number of rows of 17-month photoreceptor cells).

### DETAILED DESCRIPTION OF EMBODIMENTS

In order to make the objects, technical solutions and advantages of the examples of the present disclosure clearer, the technical solutions in the examples of the present disclosure will be described clearly and completely below. If no specific conditions are specified in the examples, they are carried out under normal conditions or conditions recommended by the manufacturers. If the manufacturers of reagents or instrument used are not specified, they are conventional products commercially available.

A construction method for an animal model with retinitis pigmentosa diseases and application in the examples of the present disclosure are described in detail below.

Hexokinae Domain Containing protein 1 (HKDC1), one of hexokinae family members, can phosphorylate glucose, and promote glucose metabolism in vivo. Whole-genome correlation experiments indicate that HKDC1 is related to gestational hyperglycemia, and bioinformatical analysis indicates that HKDC1 is highly expressed in lung cancer tissues, and it may be a new tumor treatment target spot. Moreover, inventors' preliminary studies show that Hkdc1 gene mutation is related to RP, being a great help in exploring the pathogenic mechanism of RP. Therefore, in-depth HKDC1 research has enormous potential for treatment and cause investigation of various kinds of diseases.

At present, research of Hkdc1 gene is still in an initial stage, and specific functional mechanism thereof in vivo is still unknown, restricting its development and application.

It is discovered for the first time in the present disclosure that the Hkdc1 gene is related to retinitis pigmentosa, and by knocking out the Hkdc1 gene, the animal model having typical characteristics of retinitis pigmentosa is constructed, providing to researchers the animal model for research of retinitis pigmentosa diseases and for screening a medicine for treating retinitis pigmentosa diseases, vigorously promoting scientific research, and providing a foundation for further in-depth and systematic research of pathogenic mechanism of retinitis pigmentosa diseases.

On this basis, in one aspect, the present disclosure provides a construction method for an animal model with retinitis pigmentosa diseases, which includes: knocking out a sequence of interest, which is Hkdc1 gene, on a genome of a target animal, to obtain a founder animal with the Hkdc1 gene being knocked out.

Optionally, in some embodiments of the present disclosure, the above sequence of interest is an exon sequence on the Hkdc1 gene.

Optionally, in some embodiments of the present disclosure, the above exon sequence is an exon 2 sequence.

There are a plurality of exons on the Hkdc1 gene, and this gene can be deactivated by knocking out one or more exons thereof, and is abnormally expressed, thus achieving the knockout object, and obtaining the founder animal with the Hkdc1 gene being knocked out.

Optionally, in some embodiments of the present disclosure, the above construction method further includes:

making the above founder animal with the Hkdc1 gene being knocked out and a wild-type animal of the same category to mate, to obtain a heterozygous animal with the Hkdc1 gene being knocked out; and making the above heterozygous animals with the Hkdc1 gene being knocked out to inbreed, to obtain a homozygous animal with the Hkdc1 gene being knocked out.

The homozygous animal with the Hkdc1 gene being knocked out presents typical characteristics of retinitis pigmentosa, for example, retina function damage and gradual apoptosis of photoreceptor cells, and can act as an animal model with retinitis pigmentosa diseases. This animal model can be used for research of retinitis pigmentosa diseases and for screening a medicine for treating retinitis pigmentosa diseases.

Optionally, in some embodiments of the present disclosure, the above target animal is any one selected from mouse, rat, dog, monkey and ape.

Optionally, in some embodiments of the present disclosure, the above founder animal with the Hkdc1 gene being knocked out is obtained through a following method: mixing gRNA for an Hkdc1 gene sequence with Cas9 endonuclease, injecting them to a zygote of the target animal, to obtain, after maturation, the above founder animal with the Hkdc1 gene being knocked out.

Optionally, in some embodiments of the present disclosure, a base sequence of a target site of the above gRNA is represented by SEQ ID NO:1.

The CRISPR/Cas9 gene knockout system is a gene editing technology derived from an acquired bacterial immune system developed in recent years, and upon artificial modification, it has been widely applied to research of multiple model organisms. The CRISPR-Cas9 technology has a specific DNA recognizing function, Cas9 endonuclease, guided by guide ribonucleic acid gRNA, cleaves double-stranded DNA, causing breakage of genomic double strands, and produces non-specific recombination by utilizing instability of cell genomic repair to create repairing error (insertion or deletion), thus possibly producing frameshift mutation to cause loss of gene function, and achieving the object of gene knockout.

Of course, in other embodiments, the base sequence of gRNA target site can be designed according to the sequence of interest to be knocked out, and it is not limited to the base sequence represented by SEQ ID NO:1, while taking other sequences as the target site to knock out the Hkdc1 gene also belongs to the scope of protection of the present disclosure.

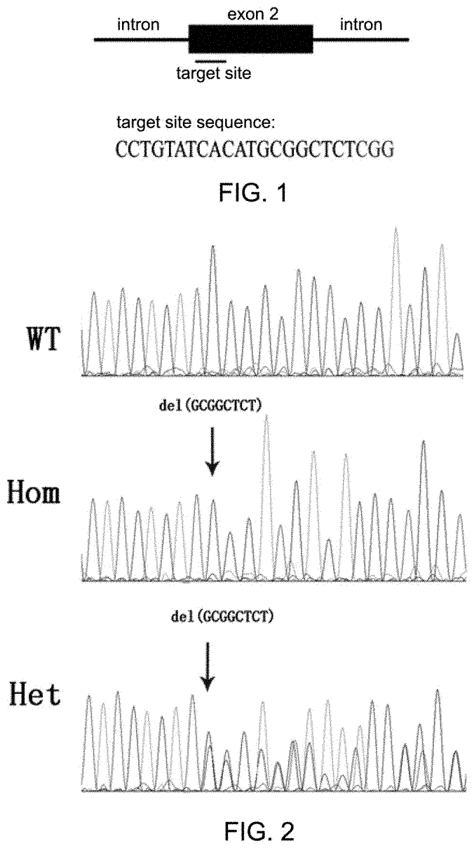

The target site represented by SEQ ID NO:1 is located at the exon 2 of the mouse Hkdc1 gene, as shown in FIG. 1.

Besides, in other embodiments, the Hkdc1 gene also can be knocked out using the Cre-loxP knockout technology, which also belongs to the scope of protection of the present disclosure.

In another aspect, the present disclosure provides application of the animal model with retinitis pigmentosa diseases obtained through construction method for the animal model with retinitis pigmentosa diseases obtained through the above construction method in screening a medicine for treating retinitis pigmentosa diseases.

In another aspect, the present disclosure provides application of the animal model with retinitis pigmentosa diseases obtained through construction method for the animal model with retinitis pigmentosa diseases obtained through the above construction method in research of retinitis pigmentosa diseases.

Optionally, in some embodiments of the present disclosure, the research is research of pathogenesis or pathogenic mechanism of retinitis pigmentosa diseases.

In another aspect, the present disclosure provides an animal model with retinitis pigmentosa diseases, which is constructed through the above construction method for an animal model with retinitis pigmentosa diseases.

In another aspect, the present disclosure provides an animal model with retinitis pigmentosa diseases, of which an Hkdc1 gene sequence on a genome thereof is knocked out.

Optionally, in some embodiments of the present disclosure, an exon sequence of the Hkdc1 gene on the genome of the animal model with retinitis pigmentosa diseases is knocked out.

Optionally, in some embodiments of the present disclosure, the animal model is any one selected from mouse, rat, dog, monkey and ape.

Optionally, in some embodiments of the present disclosure, the animal model is a mouse, and an exon 2 sequence of the Hkdc1 gene on the genome of the animal model is knocked out.

Optionally, in some embodiments of the present disclosure, compared with a wild-type mouse, 93^{rd}-99^{th} bases of cDNA sequence of the Hkdc1 gene of the animal model have deletion mutation, and a deleted base sequence is CTCTCGG.

In another aspect, the present disclosure provides a method for screening a medicine for preventing or treating retinitis pigmentosa diseases, which includes:

applying a to-be-screened candidate reagent for preventing or treating retinitis pigmentosa diseases to the animal model with retinitis pigmentosa diseases mentioned in the above.

Optionally, in some embodiments of the present disclosure, if, after the candidate reagent is applied, one or more following phenomena are detected on the animal model with retinitis pigmentosa diseases, it is indicated that this candidate reagent can be taken as a medicine for preventing or treating retinitis pigmentosa diseases:

Phenomena (1): compared with the animal model with retinitis pigmentosa diseases to which this candidate reagent is not applied, the animal model with retinitis pigmentosa diseases to which this candidate reagent is applied present reduced rhodopsin proteins accumulated in inner segment and soma of a photoreceptor cell;

Phenomena (2): compared with the animal model with retinitis pigmentosa diseases to which this candidate reagent is not applied, the animal model with retinitis pigmentosa diseases to which this candidate reagent is applied present increased thickness of retina.

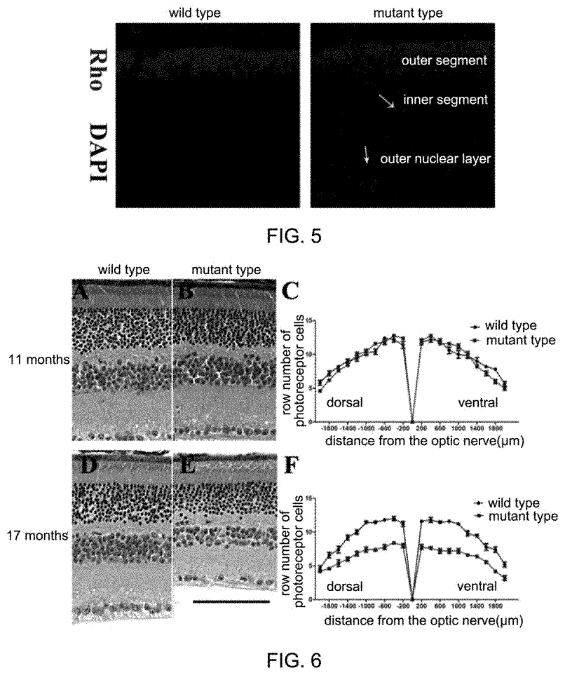

It can be seen from results of experiment examples of the present disclosure that transportation of the retinal rhodopsin proteins of the homozygous mouse with the Hkdc1 gene being knocked out (KO) is abnormal: the retinal rhodopsin proteins are accumulated in inner segment and soma of the photoreceptor cell (FIG. 5). Then, if, after a certain candidate reagent is applied, the rhodopsin proteins accumulated in inner segment and soma of the photoreceptor cell start to reduce and even disappear, it can be proved that this candidate reagent is effective on treating retinitis pigmentosa diseases, and can be taken as a medicine for preventing or treating retinitis pigmentosa diseases.

Besides, it can be seen from results of experiment examples of the present disclosure that the retina thickness of the homozygous mouse with the Hkdc1 gene being knocked out evidently becomes small: the thickness of an outer nuclear layer is evidently reduced (FIG. 6). Then, if, after a certain candidate reagent is applied, the retina thickness thereof evidently becomes big, it can be proved that this candidate reagent is effective on treating retinitis pigmentosa diseases, and can be taken as a medicine for preventing or treating retinitis pigmentosa diseases.

In another aspect, the present disclosure provides a nucleic acid molecule for constructing an animal model with retinitis pigmentosa diseases, wherein the nucleic acid molecule is gRNA, and a target sequence of the gRNA is located on Hkdc1 gene of a target animal.

Preferably, the target sequence is located on an exon of the Hkdc1 gene of the target animal;

Preferably, the target animal is any one selected from mouse, rat, dog, monkey and ape.

In some embodiments of the present disclosure, the target animal is a mouse, and the target sequence of the gRNA is represented by SEQ ID NO:1. In a further aspect, the present disclosure further provides a nucleic acid molecule for constructing an animal model with retinitis pigmentosa diseases, wherein the nucleic acid molecule is gRNA, and a base sequence of a target site thereof is represented by SEQ ID NO:1.

The nucleic acid molecule of SEQ ID NO:1 is a target site, and taking this site as a target spot, part of the sequences of the Hkdc1 gene of the target animal, for example, a mouse, can be knocked out with the help of the CRISPR-Cas9 technology, so as to obtain the animal model with retinitis pigmentosa diseases.

In another aspect, the present disclosure further provides another construction method for an animal model with retinitis pigmentosa diseases, which includes: inhibiting or silencing expression of Hkdc1 gene of a target animal.

Results of experiment examples according to the present disclosure show that the animal model with retinitis pigmentosa diseases can be obtained as long as the Hkdc1 gene expression of the target animal is inhibited or silenced through suitable methods, and these suitable methods may be, for example, knocking out the Hkdc1 gene at gene level, inhibiting transcription of the Hkdc1 gene or inhibiting translation of transcriptional mature mRNA at RNA level using siRNA, or neutralizing Hkdc1 proteins using Hkdc1 antibodies at protein level, and all of these methods can result in inactivation or deficiency of Hkdc1 proteins in the target animal, and further the animal model with retinitis pigmentosa diseases can be obtained. Therefore, all methods for inhibiting or silencing the Hkdc1 gene expression of the target animal belong to the scope of protection of the present disclosure, and animal models obtained through these methods also fall into the scope of protection of the present disclosure.

Preferably, the target animal is any one selected from mouse, rat, dog, monkey and ape.

Features and performances of the present disclosure are further described in detail below in combination with examples.

### EXAMPLES

In the present example, an animal model with retinitis pigmentosa diseases was constructed by knocking out mouse Hkdc1 gene using the CRISPR-Cas9 technology, specifically as follows.

1. Designing a gRNA sequence for targeted knockout of Hkdc1 gene, to obtain an F0 generation mouse with the Hkdc1 gene being knocked out

For an exon 2 region of the mouse Hkdc1 gene, a gRNA target site sequence was designed as follows:

(SEQ ID NO. 1)
5′-CCTGTATCACATGCGGCTCTCGG-3′.

RNA was synthesized in vitro, and micro-injected together with Cas9 endonuclease into a mouse zygote, to obtain, after maturation, a founder mouse with gene mutation.

A result schematic diagram for the gRNA sequences to target the Hkdc1 gene is as shown in FIG. 1.

2. Sequencing and verifying result of Hkdc1 gene knockout

Using the mouse genomic DNA, fragments near the present CRISPR target spot position were sequenced, to determine frameshift mutation of the founder mouse affecting a reading frame.

It was Specifically as Follows:

2.1. Extracting samples for amplification

(1) cutting a few tissue samples of tail tip of the founder mouse obtained in step 1, and disposing the samples in a clean 1.5 ml centrifuge tube;

(2) adding 100 μl of lysate (40 mM NaOH, 0.2 mM EDTA solution) to the centrifuge tube, and heating the centrifuge tube in a metal bath at 100° C. for 1 h; and

(3) taking out the centrifuge tube, after cooling the centrifuge tube to a room temperature, adding 100 μl of neutralization solution (40 mM Tris-HCl, pH 5.5), and after centrifugation of 10000 g for 2 min, taking supernatant to identify mouse genotype.

2.2. Identifying mouse genotype

(1) PCR amplification: configuring a PCR reaction system according to a following system

| | |
|---|---|
| 2 × Taq Mix: | 10 μL |
| Tail tissue lysate: | 2 μL |
| Primer 1 Hkdc1-Forward: | 1 μL |
| Primer 2 Hkdc1-Reverse: | 1 μL and |
| ddH₂O: | 6 μL |

In the above,

A Hkdc1-Forward primer base sequence was as follows:

5′-TGGTTAGGAACACATAGAACAAA-3′;

A Hkdc1-Reverse primer base sequence was as follows:

5′-CTAAGGGGCTGGTATGGGAAT-3′.

PCR reaction was carried out according to following reaction conditions:

| | |
|---|---|
| a first step 95° C. | 5 min; |
| a second step 95° C. | 20 s; |
| a third step 58° C. | 20 s; |
| a fourth step 72° C. | 30 s; |
| a fifth step | repeating the second to fourth steps 34 times; |
| a sixth step 72° C. | 5 min; and |
| a seventh step | preserving at 4° C. |

(2) Purification:

Amplified PCR fragments needed to be purified using FastAP (excision enzyme, reaction buffer and ddH₂O had been added) so as to remove the primers and other interference fragments, and a specific purification system was:

| | |
|---|---|
| PCR fragments: | 4.5 μL; and |
| FastAP: | 1.5 μL. |

Reaction conditions were:

| | | |
|---|---|---|
| step 1. | 37° C. | 30 min; |
| step 2. | 80° C. | 15 min; and |
| step 3. | 12° C. | for preservation. |

(3) Sequencing

The purified fragments having undergone the above steps subsequently were used for a sequencing reaction. A specific sequencing reaction system was:

| | |
|---|---|
| 5 × sequencing buffer: | 1.75 μL; |
| Primer-F or Primer-R (5 mM): | 0.64 μL; |
| Purified product: | 2 μL; |
| BigDye: | 0.36 μL; and |
| ddH₂O: | 5.25 μL. |

Sequencing reaction conditions were as follows:

| | | |
|---|---|---|
| step 1. | 96° C. | 30 s; |
| step 2. | 50° C. | 15 s; |
| step 3. | 60° C. | 4 min; |
| step 4. | returning back to step 1 | repeating 29 times; and |
| step 5. | 12° C. | preserving. |

After the sequencing reaction was ended, 50 μL of 70% ethyl alcohol was added to each well, followed by centrifugation of 12,000×g for 30 min, such that the DNA amplified fragments were deposited at bottom of a 96-well plate, subsequently a cover was opened, a supernatant liquid was poured out slowly, then the 96-well plate was upside-down, subjected to centrifugation of 1,000×g for 1 min, opened and placed in a light-tight condition for about 30 min such that the ethyl alcohol thoroughly volatilized, then 10 μL of ddH₂O was added so as to re-dissolve the DNA fragments, for final machine sequencing.

See FIG. 2 for frameshift mutation results obtained from the sequencing.

Mut: NM_145419.1, c.93_99deICTCTCGG; it can be seen that compared with the cDNA sequence of wild-type mouse Hkdc1 gene, 93^{rd}-99^{th} bases, 7 bases in total, of the cDNA sequence of the Hkdc1 gene of the mouse with the Hkdc1 gene being knocked out were deleted, and a deleted sequence was CTCTCGG.

3. Obtaining F1 generation mouse with the Hkdc1 gene being knocked out

The founder mouse with mutation was mated with a wild-type mouse, to obtain an F1 generation heterozygous mouse (+/−) with gene mutation, and DNA sequencing was carried out to confirm that the frameshift mutation in FIG. 2 occurred to the target gene so that the target protein is deactivated, achieving Hkdc1 gene knockout.

4. Obtaining F2 generation mouse with the Hkdc1 gene being knocked out

Heterozygous mice (+/−) with the same genotype were mated with each other to obtain an F2 generation homozygous mouse (−/−) with the Hkdc1 gene being knocked out. As a result, a homozygous knockout mouse (a sequencing result was shown by the homozygote in FIG. 2) having 7 base pairs (Mut: del ctctcgg) deleted on exon 2 was obtained.

5. Detecting HKDC1 Protein Situation

Expression of HKDC1 proteins in homozygous mutant mouse retina was verified respectively through western blot (WB) and immunohistochemistry.

In the above, the WB method was specifically as follows:

(1) respectively separating retinal tissues of the wild-type and mutant-type mice (i.e. homozygous mice (−/−) with the Hkdc1 gene being knocked out), disposing the retinal tissues in a 1.5 ml centrifuge tube, and adding 200 μl of protein lysate RIPA;

(2) after ultrasonication of the retinal tissues, lysing the retinal tissues on ice for 20 min;

(3) after centrifugation of 16000 g at 4° C. for 10 min, taking and transferring supernatant to another clean centrifuge tube, adding 50 μl of protein loading solution, and after mixing them well, heating them at 95° C. for 5 min;

(4) after cooling the samples, respectively taking 20 μl of the samples to undergo polyacrylamide gel electrophoresis (SDS-PAGE) at a voltage of 160 V so as to separate the proteins;

(5) after SDS-PAGE was ended, cutting a nitrocellulose membrane of a suitable size as required, laying filter paper, gel, the nitrocellulose membrane and filter paper in order, and debubbling, placing a transmembrane tank in an ice-water bath, and performing membrane transfer with a constant current of 0.28 A for 2 h;

(6) after completing the membrane transfer, rinsing the nitrocellulose membrane once with pure water, drying and labeling the nitrocellulose membrane, then sealing the nitrocellulose membrane with 8% defatted milk for 2 h;

(7) after completing the sealing, adding a certain amount of primary antibodies diluted in a certain proportion (according to instruction for use of the antibodies) in a sealing liquid, to be incubated at 4° C. overnight;

(8) recovering the primary antibodies, washing the membrane with 1×TBST buffer four times, 10 min each time, selecting suitable secondary antibodies according to source of the primary antibodies, diluting the secondary antibodies labeled by horseradish hydrogen peroxidase (HRP) with 1×TBST, and incubating the secondary antibodies on a shaker at a room temperature for 2 h;

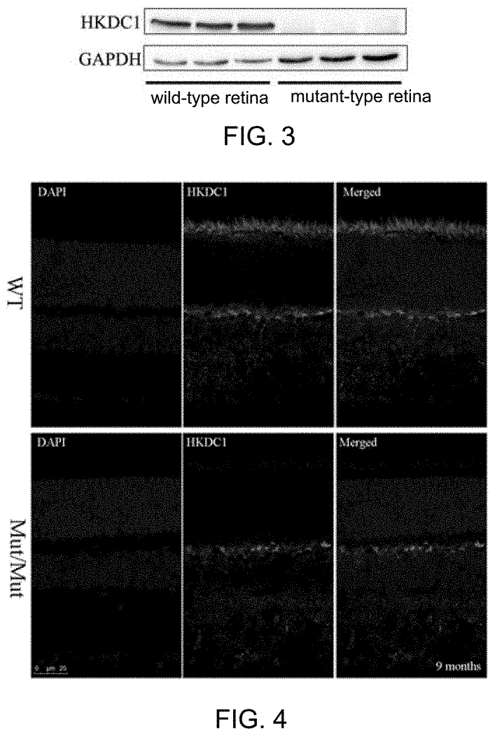

(9) after incubation of the secondary antibodies was ended, washing the membrane with 1×TBST three times, 10 min each time, detecting proteins using an ELC luminescence kit of Thermo, wherein instrument used was chemiluminescence gel imaging system of Bio-Rad. Results are as shown in FIG. 3. The results in FIG. 3 show that the HKDC1 proteins are not expressed in the mutant-type mouse retinal tissues.

An immunohistochemistry method was specifically as follows:

After killing the mouse by cervical dislocation, eyeballs were quickly taken, and placed in 4% PFA. After fixing the eyeballs on ice for 15 min, a cut was made on each cornea, and then the eyeballs were kept on being fixed on the ice. After 2 h, the eyeballs were rinsed with a PBS buffer three times, then the eyeballs were disposed in 30% sucrose solution to dewater for 2 h, then corneas and crystals were cut off under anatomical lens, embedded in OCT and quickly disposed in a −80° C. refrigerator to be frozen. After about 10 min, the OCT-embedded eyeballs were taken out, disposed on a freezing microtome to be balanced at −25° C. for about 30 min, and then sectioned, with a section thickness of 12 μm.

After completing the sectioning, sections of a relatively high quality were selected and disposed in a 37° C. oven for 30 min, then circled with a pap pen at places having retinal tissues. The sections were washed with PBS three times to remove OCT, then sealed with 5% NDS (containing 0.25% Triton) to permeate for 2 h, and the primary antibodies were incubated at 4° C. overnight. The next day, after washing with PBS twice, corresponding fluorescent secondary antibodies were incubated, then washed with PBS twice, and the sections were sealed and observed.

Results are as shown in FIG. 4. The results in FIG. 4 show that no fluorescence was observed in the mutant-type mouse retinal tissues, indicating that the HKDC1 proteins were not expressed.

It can be seen from combination of results in FIG. 3 and FIG. 4 that the HKDC1 to proteins were no longer expressed in the homozygous mutant mouse retina.

Therefore, using the gRNA sequences designed in the present disclosure, Cas9 can be guided to quickly, efficiently, and specifically knock out the Hkdc1 gene of the mouse, and the Hkdc1+/− mouse model (heterozygous type) and Hkdc1−/− mouse model (homozygous type) with the Hkdc1 gene being knocked is out can be constructed using the method of the present disclosure, and both mouse models can be used for subsequent functional research.

Test Example

Characteristics of retinal degenerative diseases presented by the mice with the Hkdc1 gene being knocked out obtained in Example 1 are described specifically below through the test example.

Research of retinal degenerative diseases by using the mouse model with the Hkdc1 gene being knocked out constructed in Example 1 1. Retinas of knockout mice were subjected to frozen sectioning, and immunohistochemical staining (an immunohistochemical method was the same as that in Example 1) is used to analyze, and it was found that when being 9 months old, transportation of retinal rhodopsin proteins of homozygous mouse with the Hkdc1 gene being knocked out (KO) was abnormal: the retinal rhodopsin proteins were accumulated in inner segment and soma of photoreceptor cells (FIG. 5). The rhodopsin is a main photoreceptor protein, being extremely crucial for vision formation, and its fault positioning will cause photoreceptor cell apoptosis, therefore, it can be seen that the Hkdc1 gene plays an important role in retinal degenerative diseases.

2. H&E staining result showed gradual apoptosis of the retinal photoreceptor cells of KO mice (homozygous mice with the Hkdc1 gene being knocked out)

Retinas of 11-month-old and 17-month-old mice were subjected to paraffin sectioning, and staining with a hematoxylin-eosin staining method (H&E staining method) through following specific operations:

1) quickly taking eyeball tissues of the mice, and disposing the eyeball tissues in stationary liquid to be fixed for 24 h;

2) embedding the eyeball tissues in paraffin, and sectioning the eyeball tissues, with a thickness of 4 μm;

3) conventionally dewaxing the sections with xylene, in multilevel of ethyl alcohol till water washing: xylene (I) for 5 min→xylene (II) for 5 min→100% ethyl alcohol for 2 min→>95% ethyl alcohol for 1 min→>80% ethyl alcohol for 1 min→75% ethyl alcohol for 1 min→distillation and water washing for 2 min;

4) staining the sections with hematoxylin for 5 minutes, and rinsing the sections by tap water;

5) differentiating the sections with hydrochloric alcohol for 30 seconds; 6) immersing the sections in tap water for 15 minutes; 7) disposing the sections in eosin for 2 minutes; 8) conventionally dewatering, clearing, and sealing the sections: 95% ethyl alcohol (I) for 1 min→95% ethyl alcohol (II) for 1 min→100% ethyl alcohol (I) for 1 min→100% ethyl alcohol (II) for 1 min→xylene carbolic acid (3:1) for 1 min→xylene (I) for 1 min→xylene (II) for 1 min→neutral resin sealing.

9) taking photographs under a microscope.

As shown in FIG. 6, it was found from the results that when being 11 months old, the WT (wild) and KO mice had no significant change in retina thickness, however, when being 17 months old, the retina thickness of the KO mouse apparently became small: the thickness of an outer nuclear layer was apparently reduced (FIG. 6).

It can thus be seen that the mouse model with the Hkdc1 gene being knocked out constructed using the method of the present disclosure has typical characteristics of retinal degenerative diseases, and can be used for research of retinal degenerative diseases.

To sum up, with specific targeted knockout of the nucleotide sequence of the Hkdc1 gene through the CRISPR-Cas9 technology provided in the examples of the present disclosure, Cas9 can be guided to quickly, efficiently, and specifically knock out the Hkdc1 gene of the mouse, to obtain the mouse model with retinitis pigmentosa diseases, providing a reliable animal model for research of Hkdc1 gene related diseases, and having broad application prospect.

The above-mentioned are merely for preferred examples of the present disclosure, and not intended to limit the present disclosure. For one skilled in the art, the present disclosure may have various modifications and variations. Any amendments, equivalent replacements, improvements and so on, within the spirit and principle of the present disclosure, should be covered within the scope of protection of the present disclosure.

Industrial applicability: with the construction method for an animal model with retinitis pigmentosa diseases provided in the present disclosure, the animal model with retinitis pigmentosa diseases with HKDC1 gene being specifically knocked out in the retina can be constructed. This model presents typical characteristics of retinitis pigmentosa diseases. This model can be used in fields such as research of retinitis pigmentosa diseases and screening a medicine for treating or preventing retinitis pigmentosa diseases, providing a model foundation for further understanding the pathogenesis of retinitis pigmentosa diseases, and screening the medicine for retinitis pigmentosa diseases.

1. A construction method for an animal model with retinitis pigmentosa diseases, comprising: knocking out a sequence of interest, which is an Hkdc1 gene, on a genome of a target animal, to obtain a founder animal with the Hkdc1 gene being knocked out.

2. The construction method for an animal model with retinitis pigmentosa diseases according to claim 1, wherein the sequence of interest is an exon sequence on the Hkdc1 gene.

3. The construction method for an animal model with retinitis pigmentosa diseases according to claim 2, wherein the exon sequence is an exon 2 sequence.

4. The construction method for an animal model with retinitis pigmentosa diseases according to claim 1, wherein the construction method further comprises:
making the founder animal with the Hkdc1 gene being knocked out and a wild-type animal of a same category to mate, to obtain a heterozygous animal with the Hkdc1 gene being knocked out; and
making the heterozygous animals with the Hkdc1 gene being knocked out to inbreed, to obtain a homozygous animal with the Hkdc1 gene being knocked out.

5. The construction method for an animal model with retinitis pigmentosa diseases according to claim 1, wherein the target animal is any one selected from the grouping consisting of mouse, rat, dog, monkey and ape.

6. The construction method for an animal model with retinitis pigmentosa diseases according to claim 1, wherein the founder animal with the Hkdc1 gene being knocked out is obtained through a following method: mixing gRNA for an Hkdc1 gene sequence with Cas9 endonuclease, and injecting them to a zygote of the target animal, to obtain, after maturation, the founder animal with the Hkdc1 gene being knocked out.

7. The construction method for an animal model with retinitis pigmentosa diseases according to claim 6, wherein a base sequence of a target site to which the gRNA is directed is represented by SEQ ID NO:1.

8. (canceled)

9. (canceled)

10. (canceled)

11. (canceled)

12. (canceled)

13. (canceled)

14. (canceled)

15. (canceled)

16. (canceled)

17. (canceled)

18. A nucleic acid molecule for constructing an animal model with retinitis pigmentosa diseases, wherein the nucleic acid molecule is gRNA, and a target sequence of the gRNA is located on an Hkdc1 gene of a target animal,
the target sequence is located on an exon of the Hkdc1 gene of the target animal; and
the target animal is any one selected from mouse, rat, dog, monkey and ape.

19. The nucleic acid molecule for constructing an animal model with retinitis pigmentosa diseases according to claim 18, wherein the target animal is a mouse, and the target sequence of the gRNA is represented by SEQ ID NO:1.

20. (canceled)

21. The construction method for an animal model with retinitis pigmentosa diseases according to claim 2, wherein the construction method further comprises:
making the founder animal with the Hkdc1 gene being knocked out and a wild-type animal of a same category to mate, to obtain a heterozygous animal with the Hkdc1 gene being knocked out; and
making the heterozygous animals with the Hkdc1 gene being knocked out to inbreed, to obtain a homozygous animal with the Hkdc1 gene being knocked out.

22. The construction method for an animal model with retinitis pigmentosa diseases according to claim 3, wherein the construction method further comprises:
making the founder animal with the Hkdc1 gene being knocked out and a wild-type animal of a same category to mate, to obtain a heterozygous animal with the Hkdc1 gene being knocked out; and
making the heterozygous animals with the Hkdc1 gene being knocked out to inbreed, to obtain a homozygous animal with the Hkdc1 gene being knocked out.

23. The construction method for an animal model with retinitis pigmentosa diseases according to claim 2, wherein the target animal is any one selected from the grouping consisting of mouse, rat, dog, monkey and ape.

24. The construction method for an animal model with retinitis pigmentosa diseases according to claim 3, wherein the target animal is any one selected from the grouping consisting of mouse, rat, dog, monkey and ape.

25. The construction method for an animal model with retinitis pigmentosa diseases according to claim 4, wherein the target animal is any one selected from the grouping consisting of mouse, rat, dog, monkey and ape.

26. The construction method for an animal model with retinitis pigmentosa diseases according to claim 2, wherein the founder animal with the Hkdc1 gene being knocked out is obtained through a following method: mixing gRNA for an Hkdc1 gene sequence with Cas9 endonuclease, and injecting them to a zygote of the target animal, to obtain, after maturation, the founder animal with the Hkdc1 gene being knocked out.

27. The construction method for an animal model with retinitis pigmentosa diseases according to claim 3, wherein the founder animal with the Hkdc1 gene being knocked out is obtained through a following method: mixing gRNA for an Hkdc1 gene sequence with Cas9 endonuclease, and injecting them to a zygote of the target animal, to obtain, after maturation, the founder animal with the Hkdc1 gene being knocked out.

28. The construction method for an animal model with retinitis pigmentosa diseases according to claim 4, wherein the founder animal with the Hkdc1 gene being knocked out is obtained through a following method: mixing gRNA for an Hkdc1 gene sequence with Cas9 endonuclease, and injecting them to a zygote of the target animal, to obtain, after maturation, the founder animal with the Hkdc1 gene being knocked out.

29. The construction method for an animal model with retinitis pigmentosa diseases according to claim 5, wherein the founder animal with the Hkdc1 gene being knocked out is obtained through a following method: mixing gRNA for an Hkdc1 gene sequence with Cas9 endonuclease, and injecting them to a zygote of the target animal, to obtain, after maturation, the founder animal with the Hkdc1 gene being knocked out.
